(19) United States
(12) Patent Application Publication
(10) Pub. No.: US 2011/0190498 A1
(43) Pub. Date: Aug. 4, 2011

(54) STEREO-SPECIFIC HYDROXYLATION

(76) Inventors: Erwin A Galinski, Senden (DE); Marlene Stein, Bonn (DE); Andrea Ures, Bitburg (DE); Thomas Schwarz, Leichlingen (DE)

(21) Appl. No.: 12/741,293

(22) PCT Filed: Nov. 7, 2008
(86) PCT No.: PCT/EP08/09414

§ 371 Date: Aug. 27, 2010

(30) Foreign Application Priority Data
Nov. 7, 2007 (DE) 10 2007 052 900.9

(51) Int. Cl. C07D 239/02; C12P 7/40; C12P 11/00

(52) U.S. Cl. 544/298; 435/136; 435/130

(57) ABSTRACT

The invention relates to a method for the stereospecific hydroxylation of α-amino-S-carboxylic acid derivatives or α-amino-R-sulfonic acid derivatives to form α-amino-β-hydroxy-S-carboxylic acid- or α-amino-β-hydroxy-R-sulfonic acid compounds, with the hydroxyl and acid groups being trans-configured and making use of microorganisms exhibiting hydroxylase activity in a production medium to which the S-carboxylic- or R-sulfonic acid derivatives are added and hydroxylated in the microorganism in the presence of oxygen and a cosubstrate using osmotically activated transporting systems of the microorganism and subsequently released actively or passively from the microorganism so as to be obtained from the production medium.

The invention relates to a method for the stereospecific trans-hydroxylation of α-amino-S-carboxylic acid and α-amino-R-sulfonic acid derivatives to form α-amino-β-hydroxy-S-carboxylic acid- and α-amino-β-hydroxy-R-sulfonic acid compounds, with hydroxyl and acid groups being trans-configured using microorganisms exhibiting hydroxylase activity in a production medium.

Chemical changes brought about by performing just a few catalytic steps and with biological systems (enzymes) being involved are also known as biotrans-formations and belong to activities performed in the field of ‘white biotechnology’ which, especially as sustainability is concerned, has become a very important factor for the chemical industry. Basically, catalytic systems are preferred here that involve a very broad spectrum of substrates. In the case of oxidation/reduction reactions, as for example hydroxylation reactions, the regeneration of the cofactors required for this poses a technical problem. In case the regeneration of the cofactor can only be effected in whole-cell systems—which means in intact, living or dormant microorganisms—quite a number of difficulties have to be overcome. For one thing, this concerns the usually speed-limiting transport of the substrate into the cells and, on the other hand, the use of an economically priced substrate for the regeneration of the cosubstrate and, thirdly, the export of the product out of the cells and into the medium from which the product can eventually be won.

In stereospecific hydroxylation involving the cofactors NADH or NADPH the problem associated with cofactor regeneration is solved by introducing a second enzymatic system, for example the formate dehydrogenase for the regeneration of the cofactor in the producer strain or a so-called membrane reactor. The substrate needed for this, i.e. formate in the above example, is consumed in the reaction. Although 2-oxoglutarate-dependent dioxygenases for hydroxylation reactions with the involvement of molecular oxygen were known they could not be applied in actual practice because the required cosubstrate was too expensive to be consumed in the reaction and whole-cell systems so far have proved uneconomical as well.

WO2001/038500A2 discloses an enzyme having tetrahydropyrimidine dioxygenase activity of Streptomyces chrysomallus which was employed in vitro for the hydroxylation of ectoine to form hydroxyectoine in the presence of 2-oxoglutarate. The expression of the enzyme encoding gene sequence in Streptomyces lividans and E. coli has been mentioned. Regarding the expression of the enzyme in the living cell attention has been drawn to the requirement that the cell first has to be made permeable to create favorable conditions for the enzyme to come into contact with the tetrahydropyrimidine to be hydroxylated. However, the problem linked with the liberation of the hydroxylated product from the living cell has not been discussed.

Ectoine is a tetrahydropyrimidine produced in numerous bacteria, for example in Halomonas elongata through ectoine biosynthesis genes. The ectoine hydroxylase also present here is capable of causing ectoine to be converted into hydroxyectoine. However, only part of the ectoine that has formed is converted into hydroxyectoine, usually significantly less than 50%. Since ectoine and hydroxyectoin can only be separated at considerable expense the isolation of pure hydroxyectoine is both time consuming and costly.

A number of potential ectoine hydroxylases have been identified in the following microorganisms:

| | | Sequence |
|---|---|---|
| | | identity |
| Strain | Protein | [%] |
| H. elongata DSM 2581^{T} | ectoine hydroxylase (EctD) | 100 |
| C. satexigens DSM 3043^{T} | phytanoyl-CoA dioxygenase | 75 |
| B. parapertussis | putative L-protine 4-hydroxylase | 51 |
| B. bronchiseptica RB50 | putative L-proline 4-hydroxylase | 51 |
| S. avemitilis MA-4680 | putative ectoine hydroxylase | 50 |
| N. farcinica IFM 10152 | putative ectoine hydroxylase | 50 |
| S. coelicolor A3(2) | putative ectoine hydroxylase | 49 |
| S. chrysmallus | ectoine hydroxylase (ThpD) | 48 |
| B. clausii KSM-K16 | L-proline 4-hydroxylase | 46 |
| Dactylosporangium sp. | L-proline 4-hydroxylase | 33 |

The in-vitro biocatalysis of ectoine to form hydroxyectoine as well as of ectoine analogs to form the hydroxylated compounds with the hydroxylase of C. salexigens has been ascertained. First results indicate similar activities of the other hydroxylases.

It is thus the objective of the invention to provide a biological system that enables the stereospecific hydroxylation especially of ectoine but also of similarly structured molecules in economically significant quantities by means of a 2-oxoglutarate-dependent dioxygenase in a whole cell system. Of particular interest as target compounds here are those stemming from the substance category of compatible solutes which may be put to use in chemical, cosmetic and pharmaceutical production processes and/or products. Such a whole-cell system shall have the transporting systems required for the introduction of the reactants from the culture medium and be capable of discharging the products via trans-port systems or by other means, for example via so-called leakages.

This objective is achieved by proposing a method according to which for the stereospecific hydroxylation of α-amino-S-carboxylic acid derivatives—hereinfafter called S-carboxylic acid derivatives—or the respective α-amino-R-sulfonic acid derivatives—hereinafter called R-sulfonic acid derivatives—to form α-amino-β-hydroxy-S-carboxylic acid- or α-amino-β-hydroxy-R-sulfonic acid compounds making use of microorganisms exhibiting hydroxylase activity in a is production medium the S-carboxylic- or R-sulfonic acid derivatives are added to the production medium and hydroxylated in the microorganism in the presence of oxygen and a cosubstrate using for example osmotically activated transporting systems of the microorganism and subsequently released actively or passively from the microorganism so as to be gained from the production medium. Hydroxylation is brought about stereotactically in such a manner that the hydroxy and carboxylic acid or sulfonic acid groups are trans configured in cyclic compounds.

Preferred embodiments of the invention are the subject matter of subclaims.

Active release in this context means liberation with the aid of a transport system whereas passive release is the liberation via mechanosensitive channels or leakages.

Derivatives and compounds within the meaning of the invention are in particular the acids themselves but also their salts, esters and amides.

The inventive method is especially suited for the complete and stereoselective conversion of the reactant/educt into the hydroxylated product which can then be won from the production medium by means of standardized processes. It is to be understood that the hydroxylated product may also be won from the microorganism.

The microorganism may be one that by nature has said hydroxylase activity. In this case it must be made sure the required transporting systems for introduction and discharge of the reactant/educt and product are available and the hydroxylase gene is expressed permanently. Although in the case of extremophilic bacteria of genus Halomonas, Marinococcus and others which possess said hydroxylase activity the relevant mechanisms needed to overcome the membrane barrier are present the hydroxylase gene is not expressed permanently. For example, the permanent expression of the naturally existing hydroxylase gene may be achieved by bringing the gene by recombinatory processes genomically under the control of another naturally existing promoter or by introducing a suitable promoter into the microorganism in a manner known per se to replace the naturally existing promoter. A suitable promoter for example is the osmotically induced promoter PromA from Marinococcus halophilus or the osmotically inducible promoters of the ectoine biosynthesis gene cluster from Halomonas elongata (as well as modifications thereof) and other stress-induced promoters or stationary-phase promoters. Alternatively, the permanent expression of the naturally existing hydroxylase gene may also be brought about by bringing the gene carried on a naturally existing plasmid or on an introduced vector under the control of suitable promoters. It is to be understood that (natural) promoters recombinatorily modified by mutation may also be used.

However, a microorganism transformed with a hydroxylase gene is preferred, said microorganism being manipulated in a known manner to express the hydroxylase, for example by using an inductor. Suitable inductors are lactose or IPTG. The preferred method of inducing the hydroxylase activity is, however, by provoking a stress situation in the culturing medium, for example by bringing about temperature or osmotic changes, or otherwise by creating a deficiency situation such as one experienced, for example, during stationary phase transition.

Preferred microorganisms are those of genus Escherichia, Klebsiella, Halomorias, Bacillus, Corynebacterium and Marinococcus, especially Escherichia coli, Halomonas elongata, Bacillus subtilis, Marinococcus halophilus and Corynebacterium glutamicum. Preferably used is the hydroxylase from H. elongata DSM 2581 transferred into the target organism in a manner known per se, for example with the aid of a plasmid. Suitable plasmids are, for example, those of the pET type (e.g. pET22b(+) provided by NOVAGEN, Madison, USA), of the pBBR type (Kovach M E, Elzer P H, Hill S D, Robertson G T, Farris M A, Roop M R, Peterson K M (1995) Gene 166:175-176) and pHSG575 and derivatives (Takeshita S, Sato M, Toba M, Masahashi W, Hashimoto-Gotoh T (1987) Gene 61:63-74). The hydroxylase gene (ectD) from Halomonas elongata DSM 2581 has been described, see A. Ures et al., Ectoine Hydroxylase (ectD) from Halomonas elongata. Poster contribution for the annual conference of VAAM in Göttingen held from Sep. 25 to 28, 2005.

Microorganism E. coli BL21 (STRATAGENE, La Jolla, USA) was, inter alia, found suitable for the implementation of the inventive method. Nevertheless, the method can also be applied to other microorganisms taking in the educts via transporting systems and releasing the products into the medium, such as, for example, other proteobacteria or Gram-positive bacteria, in particular of genus Bacillus and Corynebacterium. Moreover, yeast cells, for example of genus Hansenula and Arxula, in which ectoine hydroxylase may be expressed are as well considered suitable.

All microorganisms and plasmids mentioned here are either commercially available, freely obtainable via depositories or have been described in great detail in literature.

Naturally, 2-oxoglutarate serves as cosubstrate for the dioxygenase from Halomonas elongata mentioned hereinbefore. Customary carbon sources may be employed; in particular, also glycerine may be used as very inexpensive carbon source. Regeneration is carried out in a known manner via acetyl-CoA and the citrate cycle.

Of special importance are the transporting systems of the microorganisms used; said systems must be capable of transporting the educts through the cell membrane. In the case of E. coli these are, for example, the well-characterized trans-port systems ProP and ProU which both have a very broad substrate range and are capable of transporting linear as well as cyclic molecules through the membrane barrier. For example, glycine betaine, dimethylglycine, glycine, homobetaine, proline, proline betaine, 3,4-dihydroproline, pipecoline acid, taurine, carnitine, γ-butyrobetaine, trigonelline and ectoine are transported through the cell membrane without any difficulty, see G. Gousbet et al., Pepicolic acid is an osmoprotectant for Escherichia coli taken up by the general osmoporters ProU and ProP, Microbiology 140 (1994), 2415-2422; B. Kempf et al., Uptake and synthesis of compatible solutes as microbial stress responses to high-osmolality environments, Arch. Microbiol. 170 (1998), 319-330. The efficiency of the method may be influenced if existing transport systems altogether or to some extent are deleted, mutagenized or otherwise impaired in their selectivity.

The method proposed by the present invention can be implemented in the usual way using a culturing or production medium, with batch methods and fed-batch methods as well as continuous methods being suitably applied. A continuous approach is preferred here. The hydroxylated products can be isolated in a customary manner by means of chromatographic processes, for example with the aid of column chromatography.

The S-carboxylic acid derivatives serving as starting compounds are α-amino-S-carboxylic acid derivatives having the basic structure of amino acids and thus being capable of forming zwitterions.

Especially preferred are cyclic S-carboxylic acid derivatives (and the respective R-sulfonic acid derivatives) which have an α-amino group arranged in the ring system. The ring system may have 3 to 8 members whereas aside from the nitrogen atom in the α-position to carboxylic acid one or several additional nitrogen, oxygen or sulfur atoms may be present. The ring may further be substituted by alkyl groups with up to six carbon atoms, halogen atoms, amino, imino, nitro, CN, hydroxy, ether or ester groups, with especially alkyl residues with up to six carbon atoms being suited for the ether and ester residues. Naturally, the same applies analogously to non-cyclic educts.

It has been found that the inventive method is capable of transforming the starting compounds in a highly stereospecific manner into the trans-β-hydroxy-S-carboxylic acid compounds and/or —R-sulfonic acid compounds. Said transformation is not performed enzymatically within a buffer solution external to the cell—as is the prior-art approach—but inside the cell of the transformed microorganism itself which in turn releases the product to the medium.

Contrary to other 2-oxoglutarate-dependent dioxygenases the one preferably used and stemming from Halomonas elongata is relatively less specific and capable of hydroxylating a range of starting compounds. The broad substrate spectrum is supplemented by the system's capability to transport the educt via transporting systems to the hydroxylase expressed in the cytoplasm and after transformation release the product into the medium. This causes the cell membrane to be passed through and the cofactor is regenerated by the cells themselves. Without resulting in the expressing cells to be destroyed the product can be accumulated in the medium in very high concentration and virtually any desired amount and may then be retrieved from the medium.

As mentioned above the membrane proteins (transporters) employed for conveyance within the cells are in the case of E. coli preferably the osmolyte transporters propand proU. These transporters are non-specific systems that aside from the preferred substrates glycine betaine and proline betaine also accept, inter alia, proline, ectoine and a number of ectoine derivatives. Basically, all trans-port systems are suitable that are able to overcome the membrane barrier for educts, which, for example, also includes other transporter systems for amino acids.

As has already been mentioned, the transport systems ProP and ProU from E. coli have a broad substrate system. Known transport systems of Bacillus subtills are OpuA, OpuB, OpuC and OpuD, for Corynebacterium glutamicum EctP, BetP, ProP and LcoP. All these transport systems also accept a broad substrate spectrum.

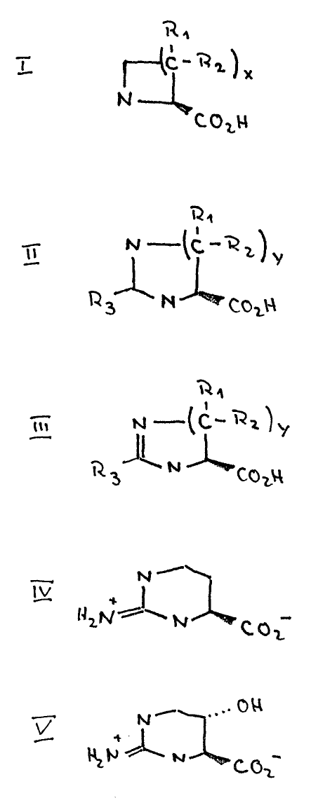

The inventive method enables in a most economical fashion the almost complete transformation of a substrate into a β-hydroxy-functionalized product which enriches itself in the medium proper. In the event of α-amino carboxylic acid derivatives and α-amino sulfonic acid derivatives the method always brings about a stereo configuration with the hydroxyl group in trans-position to the carboxylic acid or sulfonic acid group. Some products have been shown in FIG. 1.

Among the substances named hydroxyectoine and similar derivatives are of special interest because these may be put to use in particular as novel cell-protecting substances for cosmetic applications and medicinal products. For example, novel biopolymers based on hydroxylated monomers may be employed as amphoters, so-called styling polymers, in cosmetics or for the coating of surfaces with a view to improving their biocompatibility. With prior-art technologies and methods these substances cannot be produced for the mentioned applications in an economically efficient way.

### EXAMPLE

### Hydroxylation of Ectoine and the Like

The production strain E. coli BL21 contains the hydroxylase gene from Halomonas elongata on the plasmid pET22b(+)-EctD. It is cultivated in a medium having the following composition at 37° C.: 13.61 g/l KH₂PO₄, 4.21 g/l KOH, 1.98 g/l (NH₄)SO₄, 0.25 g/l MgSO₄×7 H₂O, 1.1 mg/l FeSO₄×7 H₂O, 10 g/l NaCl 5 g/l glucose, pH 7.0 adjusted with KOH, carbenicillin 100 mg/l.

The medium was aerated with oxygen, with oxygen saturation being kept at more than 50% by variation of the flow rate of the supplied compressed air (0.1 to 0.5 l/min) and the agitator speed (200 to 600 rpm). When an optical density of 0.6 (at 600 nm) was reached the expression was induced by injecting 1 ml of IPTG solution (23.8 mg/ml, sterilely filtered) per liter. Roughly one hour after induction the sterile addition of the substrate took place (final concentration 2 to 20 mM), if expedient in combination with a carbon source for the regeneration of the cofactor. The hydroxylation of the substrate was verified both in the cells and in the medium by taking samples and subsequent HPLC analysis. Through the subsequent addition of substrate in combination with a C-source (e.g. glycerine) the yield could be improved and the concentration of the product in the medium increased. After the substrate had been completely converted the culture was separated from the culturing medium by centrifugation and prepared for the isolation of the product. The conversion rate came to more than 0.1 mmol/g of dry biomass and hour.

Educt and C-source have to be added simultaneously and in adequate amount. If glycerine is added as C-source the molar ratio is 1:1.

By way of example the method was implemented with standard concentrations ranging between 2 and 20 mM. Higher concentrations are possible. The oxygen saturation level is not decisive; what is important is that oxygen in sufficient amount is available to the microorganisms.

By way of example, the method was implemented using plasmid pET22b (+)-EctD. Plasmids of pBBR type and low-copy plasmids, for example pHSG 575, are also well suited, in particular if the hydroxylase gene from Halomonas elongata is under the control of an osmotically induced promoter such as for example PromA from Marinococcus halophilus, stress-induced promoters, stationary phase promoters or other efficient promoters.

Preferred educts are compounds of formulas I, II and III, in which R₁ and R₂ stand for hydrogen and R₃ being hydrogen, an alkyl group with up to 6 carbon atoms or an amino group. x stands for an integer of between 1 and 5, y for 1 to 4.

Formula IV represents guanidinium ectoine with an S-configuration on C₂. The compound corresponds to formula III with R₁═R₂═H, y=2 and R₃═NH₂.

On the product side stands R₂ for a 3-hydroxy group which is trans-positioned to the S-carboxylic group. The hydroxylation will exclusively be effected in trans-3-position. Formula V illustrates the hydroxylation product of guanidinium ectoine.

It goes without saying that the structures Ito V may be substituted by further alkyl groups with up to 6 carbon atoms, halogen atoms, amino, imino, nitro, CN, hydroxy, ether or ester groups.

Finally, the invention also relates to the new compounds 3-hydroxyhomoectoine, 3-hydroxy-DHMICA, 3-hydroxy-ADPC, 3-hydroxy guanidinium ectoine and 3-hydroxy-2-acetidine-2-carboxylic acid and their derivatives, especially esters, amides and salts. In all these compounds the hydroxy group is in trans-position to the carboxylic acid and/or sulfonic acid function. The invention further relates to the linear compounds that can be obtained through alkaline hydrolysis from the hydroxylated compounds: N-α-acetyl- and N-6-acetyl-2,5-diamino-3-hydroxy-valeric acid, N-α-acetyl- and N-β-acetyl-2,3-diamino-3-hydroxy propionic acid, 3-hydroxy glutamine as well as their derivatives, especially esters, amides and salts.

1. Method for the stereospecific hydroxylation of α-amino-S-carboxylic acid derivatives or α-amino-R-sulfonic acid derivatives to form α-amino-β-hydroxy-S-carboxylic acid- or α-amino-β-hydroxy-R-sulfonic acid compounds, with the hydroxyl and acid groups being trans-configured and making use of microorganisms exhibiting hydroxylase activity in a production medium to which the S-carboxylic- or R-sulfonic acid derivatives are added and hydroxylated in the microorganism in the presence of oxygen and a cosubstrate using osmotically activated transporting systems of the microorganism and subsequently released actively or passively from the microorganism so as to be obtained from the production medium.

2. Method according to claim 1, characterized in that the microorganism naturally has a hydroxylase gene or is a microorganism transformed with a hydroxylase gene.

3. Method according to claim 1, characterized in that the microorganism is a microorganism transformed with a transporter gene or naturally possesses a suitable transporter system.

4. Method according to claim 1, characterized in that the genes under the control of naturally existing promoters are brought to permanent expression or the microorganism is a microorganism transformed by means of a natural, mutated or genetically modified promoter.

5. Method according to claim 1 characterized in that the hydroxylase gene stems from Halomonas elongata.

6. Method according to claim 5, characterized in that the hydroxylase gene is contained in a plasmid of pET type, pBBR type or pHSG 575 as well as derivatives thereof.

7. Method according to claim 1, characterized in that the microorganism stems from genus Escherichia, Bacillus Corynebacterium, Klebsiella, Marinococcus or Halomonas.

8. Method according to claim 7, characterized in that E. coli BL 21 is used as microorganism.

9. Method according to claim 1, characterized in that the cosubstrate 2-oxoglutarate is regenerated with glycose, acetat or glycerine as C-source.

10. Continuous method according to claim 1.

11. Method according to claim 1, characterized by the chromatographic winning of the trans-β-hydroxy carboxylic- or -sulfonic acid compounds.

12. Method according to claim 1, characterized in that an α-amino-S-carboxylic acid derivative is used.

13. Method according to claim 1, characterized in that a cyclic α-amino-S-carboxylic acid derivative is used which contains an amine function integrated into the ring system.

14. Method according to claim 1, characterized in that the S-carboxylic acid derivative is a compatible solute.

15. Method according to claim 1, characterized in that the S-carboxylic acid derivative is ectoine, homoectoine, DHMICA, ADPC, proline, acetidine-2-carboxylic acid or guanidinium ectoine.

16. Method according to claim 1, characterized by the complete hydroxylation from ectoine to hydroxyectoine.

17. 3-hydroxyhomoectoine, 3-hydroxy-DHMICA, 3-hydroxy-ADPC, 3-hydroxyguanidium ectoine and 3-hydroxy-2-acetidine carboxylic acid, their esters and salts as well as linear compounds obtainable therefrom through alkaline hydrolysis.
